(19) United States
(12) Patent Application Publication
(10) Pub. No.: US 2009/0076396 A1
(43) Pub. Date: Mar. 19, 2009

(54) Optical wavelength range for high contrast imaging of cancer

(75) Inventors: Anna N. Yaroslavsky, N. Andover, MA (US); Elena Salomatina, Boston, MA (US)

Correspondence Address: EDWARDS ANGELL PALMER & DODGE LLP, P.O. BOX 55874, BOSTON, MA, 02205, US

(73) Assignee: The General Hospital Corporation, Boston, MA (US)

(21) Appl. No.: 12/283,813

(22) Filed: Sep. 16, 2008

Related U.S. Application Data
(60) Provisional application No. 60/994,038, filed on Sep. 17, 2007.

(51) Int. Cl. A61B 6/00

(52) U.S. Cl. 600/476

(57) ABSTRACT

An apparatus for imaging cancer in which cancer tissue is contrasted against adjacent healthy tissue. The apparatus includes illumination and, possibly polarization means configured to provide illumination light on a region of tissue. Detection means are optically coupled to the illumination means, the detection means being adapted to detect reflected light from the tissue (and its polarization state, if applicable) when the tissue is illuminated by the illumination means. Filtering means are optically coupled to the detection means. The filtering means are configured to filter the illumination light and/or reflected light to pass only light within a range, or any subrange between about 1050 to about 1400 nm. Methods for imaging cancer contrast cancer tissue against adjacent healthy tissue.

### CROSS-REFERENCE TO RELATED APPLICATION

This application claims priority to U.S. Provisional Patent Application No. 60/994,038 filed Sep. 17, 2007, which is incorporated herein by reference.

### BACKGROUND OF THE INVENTION

1. Field of the Invention

The present invention relates to optical imaging of cancerous and precancerous tissue. Particularly, the present invention is directed to optical imaging of dysplastic and cancer tissue within a wavelength range in which cancer tissue is contrasted with adjacent healthy tissue.

2. Description of Related Art

A variety of devices and methods are known in the art for detecting cancer tissue. Of such devices, many are directed to optical detection of cancer tissues. Nonmelanoma skin cancers including basal cell carcinomas (BCC) and squamous cell carcinomas (SCC), are more common than all other types of human cancers. These cancers have an intrinsically low optical contrast in the visible spectral range. A number of techniques and contrast agents have been suggested and tested for the detection and delineation of these tumors. So far the best results were yielded with methods that employ exogenous contrast agents. However, if a method capable of resolving subtle differences in the optical properties of endogenous chromophores of normal and cancerous tissues was available, this would be a great asset for tumor detection.

U.S. Pat. No. 5,451,785 to Faris describes a method and apparatus for direct two-dimensional transillumination imaging of a sample immersed in or including a scattering medium at infrared to near-infrared (NIR) wavelengths. The method and apparatus in Faris can be used for the detection of malignant tumors. However, the transillumination apparatus makes in vivo detection of many cancer tissues difficult or impossible because the illumination light must project from one side of the tissue, while the detection device (e.g., camera) must be on the opposite side of the tissue in order to receive the light from the illumination means through the tissue sample.

U.S. Pat. No. 5,800,350 to Coppleson, et al. describes an apparatus for identifying tissue in the context of cancer detection. The apparatus includes a probe configured to contact the tissue. The probe includes means to subject the tissue to a variety of different stimuli such as electrical, optical, thermal, acoustic, and magnetic stimulation to detect physical response to the stimuli. The probe connects to a processor that compares the responses to categorize the tissue based on a catalogue of expected tissues to identify the tissue (e.g., normal, cancerous, pre-cancerous, or unknown). However, the apparatus requires catalogued data, which may or may not be available for a given tissue type or for cancer within the tissue type. Moreover, it is not always possible or desirable to bring a probe into direct contact with tissue needing to be imaged.

U.S. Patent Application Publication No. 2005/0240107 to Alfano et al. describes a minimally invasive method for enabling detection of cancerous tissues with spectral optical imaging using key water absorption wavelengths. Water content is an important diagnostic parameter because cancerous and pre-cancerous tissues may have different water content than normal tissues. Key water absorption wavelengths include at least one of 980 nm, 1195 nm, 1456 nm, 1944 nm, 2880 nm to 3360 nm, and 4720 nm. In the range of 400 nm to 6000 nm, one or more points of negligible water absorption were used as reference points for a comparison with neighboring key water absorption wavelengths. The resulting image can be used to detect cancer tissue. However, since the apparatus measures transmitted and/or scattered light from a tissue sample, it is prone to size limitations similar to those in U.S. Pat. No. 5,451,785, described above, when imaging of in vivo tissue is required.

International Publication No. WO 2006/076810 to Zeng et al., describes a method and apparatus for measuring cancerous changes from reflectance spectral measurements obtained during endoscopic imaging. The device uses analysis of diffuse reflectance spectra measured in vivo for cancer detection. A lamp provides white light for imaging and strong blue light (400-450 nm) with a weak NIR light for fluorescence imaging and fluorescence spectral measurements. The NIR light is employed to form a NIR reflectance image used to normalize the green fluorescence image. The measured reflected diffuse spectra are analyzed using a specially developed light-transport model and numerical method to derive quantitative parameters (such as absorption and scattering coefficients) related to tissue physiology and morphology. However, this technique requires use of a spectrometer, which adds bulk and cost to the system. In addition, the light penetration depth at NIR wavelengths is much (2-3 times) greater than at 400-450 nm. This implies that the probing depths (or the measured volumes) differ significantly. Therefore, normalization of the green fluorescence images using NIR reflectance is not accurate.

Such conventional methods and systems generally have been considered satisfactory for their intended purpose. However, many of the known methods are difficult or impossible to apply to in vivo imaging of cancer tissue. Although solutions to this problem have been developed, such as using the endoscopic spectrometer in WO 2006/076810, there still remains a continued need in the art for a method and system that allow for high-contrast imaging of cancerous tissue in vivo. There also remains a need in the art for a system of optical imaging of cancerous tissue that is inexpensive and easy to make and use. The present invention provides a solution for these problems.

### SUMMARY OF THE INVENTION

The purpose and advantages of the present invention will be set forth in and become apparent from the description that follows. Additional advantages of the invention will be realized and attained by the methods and systems particularly pointed out in the written description and claims hereof, as well as from the appended drawings.

To achieve these and other advantages and in accordance with the purpose of the invention, as embodied herein, the invention includes an apparatus for imaging cancer in which cancer tissue is contrasted against adjacent healthy tissue. The apparatus includes illumination means configured and adapted to provide illumination light on a region of tissue. Detection means are optically coupled to the illumination means. The detection means are configured and adapted to detect light reflected from the tissue and, if necessary its polarization, when the tissue is illuminated by the illumination means. Filtering means are optically coupled to the detection means. The filtering means are configured and adapted to filter at least one of the illumination light and the reflected light to pass only light within a range of about 1050 and about 1400 nm.

It is also contemplated that the filtering means and illumination means can be combined as an illumination means having a narrow band of illumination, such as a gas or diode laser, a narrow band LED, or any other suitable device.

In accordance with another aspect of the invention, the filtering means is configured to pass only light within a wavelength range in which cancerous tissue and healthy tissue have absorption coefficients that are approximately equal, and in which cancerous (or pre-cancerous) tissue has a scattering coefficient different from that of healthy tissue, causing cancerous tissue to appear darker or brighter than healthy tissue. The filtering means can be configured and adapted to filter at least one of the illumination light and the reflected light to pass only light within a range of about 1050 and about 1400 nm, including any of the subranges within the above mentioned range.

It is also envisioned that the illumination means and detection means can be combined in a confocal imaging section or polarization imaging section. At least one flexible optical coupling element can be optically coupled between the confocal imaging section and a remote probe that is insertable into a body. The at least one flexible optical coupling element can be a coherent bundle of optical fibers. It is also possible that the at least one flexible optical coupling element can be an incoherent bundle of optical fibers, wherein the imaging section includes means for descrambling the reflected light received through the incoherent bundle to form an image. The filtering means can be combined with the illumination means as a narrow band laser, or other suitable device.

The invention also includes a microscope for imaging cancer in which cancer tissue is contrasted against adjacent healthy tissue. The microscope includes at least one probe section configured and adapted to illuminate a region of tissue of interest, an illuminating section for generating illumination light for the at least one probe section, an imaging section for constructing images from reflected light remitted from the region of interest, and filtering means in optical communication with the imaging section. The filtering means are configured and adapted to filter the illumination light and the reflected light to pass only light in the optical bandwidth between about 1300 to about 1350 nm. At least one flexible optical coupling element is optically coupled between the imaging section and the probe section.

In accordance with yet another aspect of the invention, the microscope is a confocal microscope wherein the illumination section and the imaging section are combined into a confocal imaging section for generating illumination light for at least one probe section and for constructing images from reflected light remitted from the region of interest. It is also envisioned that the probe section can be a remote probe insertable into locations within a body in place of an endoscope. The at least one flexible optical coupling element can be a coherent bundle of optical fibers. It is also contemplated that the at least one flexible optical coupling element is an incoherent bundle of optical fibers wherein the imaging section includes means for descrambling the reflected light from the incoherent bundle to form an image. The filtering means can be combined with the illumination section as a narrow band gas laser, narrow band diode laser, tunable laser, narrowband LED, or any other suitable device. It is possible to use a bandpass filter, narrow band filter, monochroimator, interference filter, liquid crystal filter, or any other suitable device.

The invention further includes a method of imaging cancer in which cancer tissue is contrasted against adjacent healthy tissue. The method includes illuminating a region of tissue with illumination light, filtering at least one of the illumination light and light reflected from the region such that the light is in the optical bandwidth between about 1050 and about 1400 nm, or any subrange of this range (including monochromatic light), and registering an image based on the reflected light.

In accordance with another aspect of the invention, the filtering step includes passing only light within a wavelength range in which cancerous tissue and healthy tissue have absorption coefficients that are approximately equal, and in which cancerous tissue has a higher or lower scattering coefficient than healthy tissue, causing cancerous tissue to appear darker or brighter than healthy tissue. It is also contemplated that the steps of illuminating and constructing an image can be performed in a confocal imaging section of a confocal microscope. The step of illuminating can include transmitting illumination light between the confocal imaging section and a remote probe that is insertable into a body. It is also possible for the step of illuminating to include polarizing the illumination light, and for the registering step to include registering two reflected polarization components to be processed into an image.

It is to be understood that both the foregoing general description and the following detailed description are exemplary and are intended to provide further explanation of the invention claimed. The accompanying drawings, which are incorporated in and constitute part of this specification, are included to illustrate and provide a further understanding of the method and system of the invention. Together with the description, the drawings serve to explain the principles of the invention.

### BRIEF DESCRIPTION OF THE DRAWINGS

The patent or application file contains drawings executed as photographs in color and black and white. Copies of this patent or patent application publication with color drawings will be provided by the Office upon request and payment of the necessary fee.

FIG. 1 is a graph showing absorption and scattering coefficients for epidermis over a range of wavelengths from 400 nm to 1600 nm, averaged over 7 samples.

FIG. 2 is a graph showing absorption and scattering coefficients for dermis over a range of wavelengths from 400 nm to 1600 nm, averaged over 8 samples.

FIG. 3 is a graph showing absorption and scattering coefficients for subcutaneous fat over a range of wavelengths from 400 nm to 1600 nm, averaged over 10 samples.

FIG. 4 is a graph showing absorption and scattering coefficients for nodular basal cell carcinomas (BCC) over a range of wavelengths from 400 nm to 1600 nm, averaged over 5 samples.

FIG. 5 is a graph showing absorption and scattering coefficients for infiltrative BCC over a range of wavelengths from 400 nm to 1600 nm, averaged over 6 samples.

FIG. 6 is a graph showing absorption and scattering coefficients for squamous cell carcinomas (SCC) over a range of wavelengths from 400 nm to 1600 nm, averaged over 8 samples.

FIG. 7a is a graph showing statistical significance in the difference between absorption properties of healthy and cancerous epidermis as a function of wavelength for epidermis having infiltrative BCC, nodular BCC, and SCC.

FIG. 7b is a graph showing statistical significance in the difference between scattering properties of healthy and cancerous epidermis as a function of wavelength for epidermis having infiltrative BCC, nodular BCC, and SCC.

FIG. 8a is a graph showing statistical significance in the difference between absorption properties of healthy and cancerous dermis as a function of wavelength for dermis having infiltrative BCC, nodular BCC, and SCC.

FIG. 8b is a graph showing statistical significance in the difference between scattering properties of healthy and cancerous dermis as a function of wavelength for dermis having infiltrative BCC, nodular BCC, and SCC.

FIG. 9a is a graph showing statistical significance in the difference between absorption properties of healthy and cancerous subcutaneous fat as a function of wavelength for subcutaneous fat having infiltrative BCC, nodular BCC, and SCC.

FIG. 9b is a graph showing statistical significance in the difference between scattering properties of healthy and cancerous subcutaneous fat as a function of wavelength for subcutaneous fat having infiltrative BCC, nodular BCC, and SCC.

FIG. 10 is a confocal image a) of nodular BCC in the range between 1040 nm and 1400 nm and corresponding image b) showing frozen haematoxylin and eosin stain (H&E) histopathology for comparison.

FIG. 11 is a confocal image a) of infiltrative BCC in the range between 1040 nm and 1400 nm and corresponding image b) showing frozen H&E histopathology for comparison.

FIG. 12 is a confocal image a) of nodular BCC and a hair follicle in the range between 1040 nm and 1400 nm and corresponding image b) showing frozen H&E histopathology for comparison.

FIG. 13 is a confocal image a) of epidermis and adjacent dermis in the range between 1040 nm and 1400 nm and corresponding image b) showing frozen H&E histopathology for comparison.

FIG. 14 is a schematic view of an exemplary embodiment of an apparatus for imaging cancer in accordance with the present invention.

FIG. 15 is a table showing spectral regions of maximal optical contrast between normal and cancerous tissues for samples of epidermis, dermis, and subcutaneous fat with infiltrative BCC, nodular BCC, and SCC.

### DETAILED DESCRIPTION OF THE PREFERRED EMBODIMENTS

Reference will now be made in detail to the present preferred embodiments of the invention, examples of which are illustrated in the accompanying drawings. The method and corresponding steps of the invention will be described in conjunction with the detailed description of the system. The devices and methods presented herein may be used for imaging and diagnosing cancer tissues. The present invention is particularly suited for in vivo optical imaging of cancerous tissues in which cancerous tissue is contrasted with adjacent healthy tissue.

Differences in absorption and/or scattering of cancerous and normal skin have the potential to provide a basis for non-invasive cancer detection. In accordance with the invention, it was discovered that there are significant differences in the scattering of cancerous and healthy tissues in the spectral range from 1050-1400 nm. In this spectral region, the scattering dominates the absorption by at least one order of magnitude, the scattering of cancerous lesions is consistently lower than that of normal tissues, and absorption does not differ significantly, with the exception of nodular BCC. Nodular BCC exhibits significantly lower absorption as compared to normal skin. Therefore, in accordance with the invention, the spectral range between about 1050 nm and about 1400 nm is optimal for cancer detection. The invention provides a method and apparatus using the range of wavelengths to provide high contrast optical imaging of cancer. This wavelength range (1040 nm-1400 nm) has been validated by experiments described below for imaging nonmelanoma skin cancers.

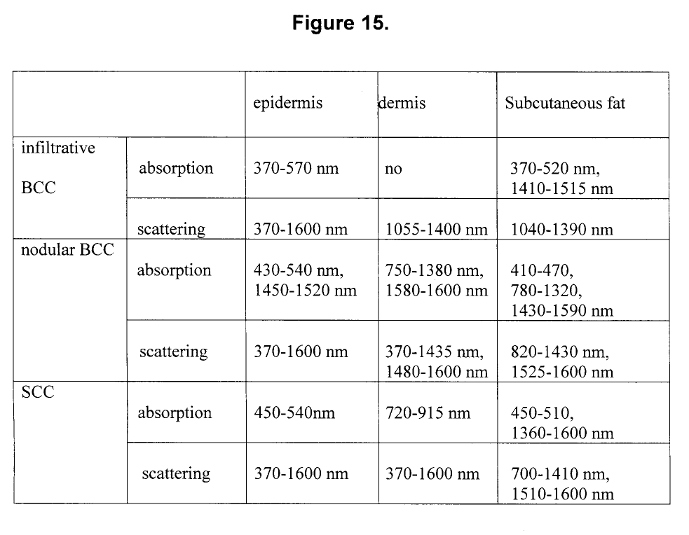

During experimental procedures in conjunction with the invention, optical properties of normal skin layers were determined and compared to those of nonmelanoma skin cancers in the wide spectral range from 370 to 1600 nm with the purpose of selecting the spectral range where the differences between cancer and normal tissue are maximal. Several wavelength regions were discovered, where the difference in absorption and scattering properties of each healthy tissue-cancer pair were statistically significant. FIG. 15 shows these wavelength regions, as described in further detail below. The results of the study indicate that there exists no spectral region within the wavelength range investigated where the differences in absorption are significant for all healthy tissue-tumor pairs simultaneously. However, all healthy tissue-cancer pairs exhibited statistically significant differences in scattering in the wavelength range from 1050 to 1400 nm. Absorption properties of normal skin layers and cancerous specimens did not differ significantly in this region with the exception of nodular BCC-normal tissue pair. Nodular BCC exhibits lower absorption as compared to normal skin. Thus, the spectral range from 1050 to 1400 nm where scattering properties of normal and cancerous tissues exhibit maximal difference may provide a reliable base for pathology discrimination.

The experiments employed integrating sphere spectrophotometry combined with the inverse Monte Carlo technique. Seven samples of human epidermis were measured and analyzed in total, eight samples of dermis, ten samples of subcutaneous fat, five samples of nodular BCC, six samples of infiltrative BCC, and eight samples of SCC. The differences in scattering and absorption between each normal and cancerous tissue type were statistically analyzed using an unpaired two-tailed t-test, and the spectral ranges where these differences were maximal identified.

Fresh specimens of normal and cancerous human skin were obtained from the surgeries under an IRB-approved protocol. The time between the surgical removal of the skin and the measurements did not exceed 7 hours. Skin excisions from the face, scalp, neck, and back of the patients were used for the experiments. The samples were briefly rinsed in Dulbecco's Phosphate-Buffered Saline (pH 7.4) solution and sectioned using a microcryotome. Sections were cut parallel to the tissue surface. The thickness of each section was measured using a high-precision digital micrometer with the accuracy of ±1 μm. The thickness of the epidermis, dermis, fat, and cancerous tissues sections varied between 60 and 100 μm, 100 and 780 μm, 280 and 800 μm, and 170 and 850 μm, respectively. The lateral size of the sectioned tissues was in the 6 to 17 mm range. Sectioned specimens were hydrated with saline and sealed between a microscopic slide and a coverslip with rapid mounting media for microscopy ENTELLAN® New drops (available from Merck in Whitehouse Station, N.J.) to prevent desiccation. In total, seven epidermis, eight dermis, ten subcutaneous fat, six infiltrative BCC, five nodular BCC, and eight SCC specimens were investigated.

For measuring total transmittance and diffuse reflectance, the light from a halogen lamp was focused onto the sample, which was mounted on the entrance and exit ports of the integrating sphere, respectively. The lateral size of the investigated sample always exceeded the diameter of the respective sphere port. The diameter of the beam on the sample did not exceed 3 mm. For the samples with lateral sizes between 6 and 7 mm, the size of the beam was reduced to 2 mm. The light diffusely reflected and transmitted by the sample was detected by two CCD-based spectrometers connected via the optical fibers to the detector ports. We have used an HR2000 spectrometer in the 370-980 nm region and an EPP2000-NIR InGaAs spectrometer in the 900-1600 nm spectral range. The wavelength calibration of the spectrometers was performed using an HgAr calibration lamp with an accuracy better than 1 nm. For all the measurements, a signal-to-noise ratio was not worse than 200:1. To ensure that the same area of the sample was examined, all the slides were marked with a permanent marker around the investigated area during the first measurement.

An inverse Monte Carlo technique was used to recover the optical properties of the samples from the measured quantities of diffuse reflectance and total transmittance. The technique employs a combination of a quasi-Newton inverse algorithm and a forward Monte-Carlo simulation, as are known to those skilled in the art. The inverse quasi-Newton algorithm is an iterative optimization technique that combines rapid local convergence of the Newton method with the ability to achieve proximity of the solution if the initial approximation is poor. In most practical cases, the algorithm required less than 10 iterations to converge. The forward Monte Carlo method is based on the numerical simulation of photon transport in scattering media. The algorithm takes into account exact optical and geometrical configuration of experiment, mismatch of the refractive indices on the boundaries of the sample, light losses at the edges of the sample, finite beam diameter and port dimensions of the integrating sphere, and arbitrary angular distribution of the incident light. Interpretation of all the experimental data obtained in this study was conducted under the assumption of the Henyey-Greenstein scattering phase function. The anisotropy factor, g, was assumed to be 0.8 and the refractive index of 1.4 for all the skin layers in the entire spectral range investigated.

A two-tailed t-test was used to evaluate the significance of the difference between obtained optical properties of healthy skin layers and cancerous tissues. The differences between optical coefficients of normal and cancerous tissues were considered to be statistically significant when the calculated probability value (p-value) was equal or less than 0.05. P-value ≦0.05 means that the probability that the two data-sets are different is ≧95%. Calculated p-values were plotted against the wavelength to identify the spectral regions, where the optical properties of normal and cancerous tissues differed significantly.

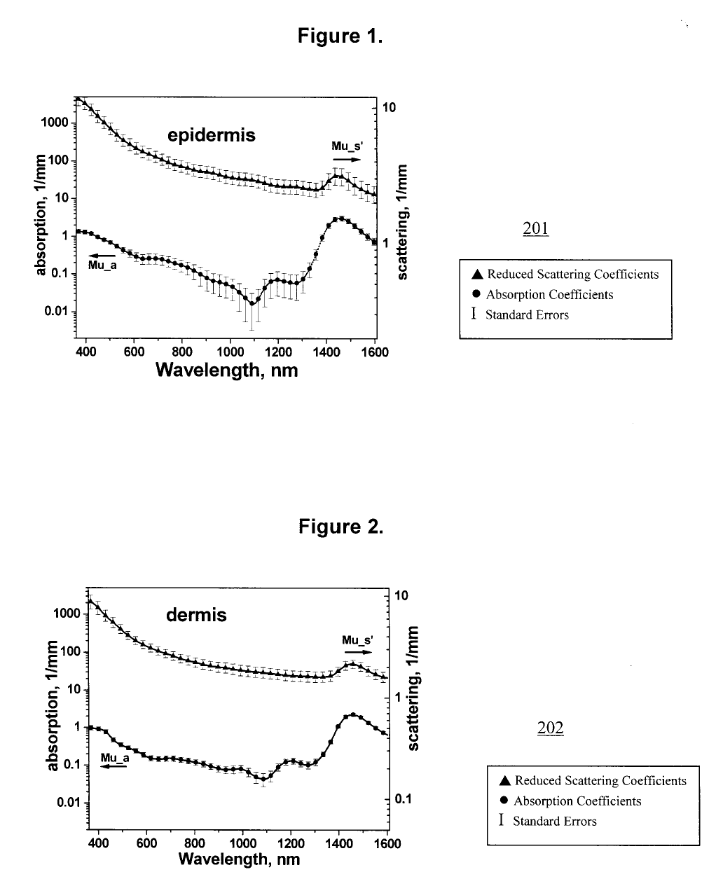

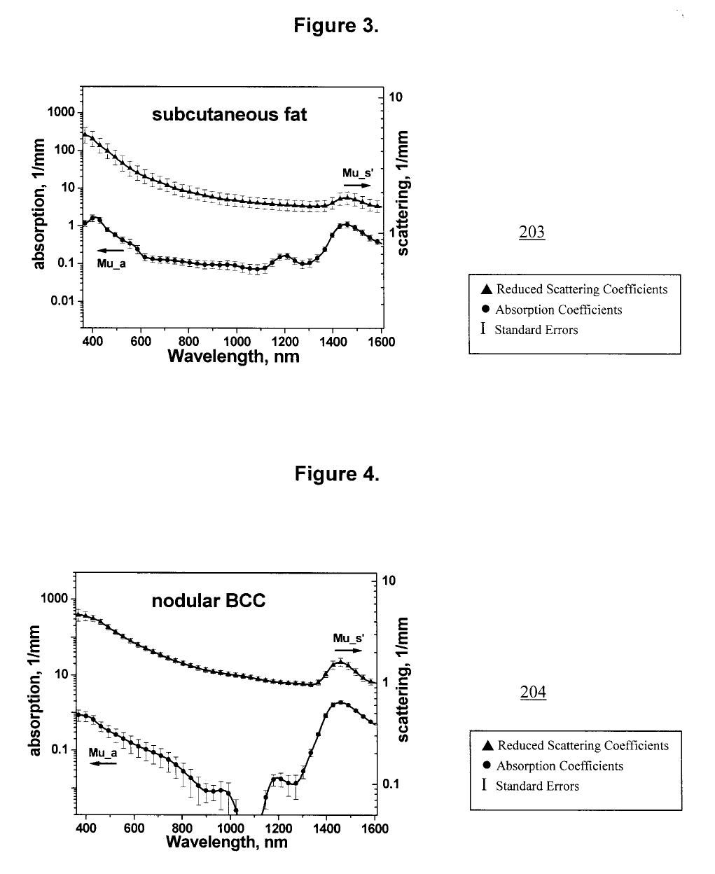

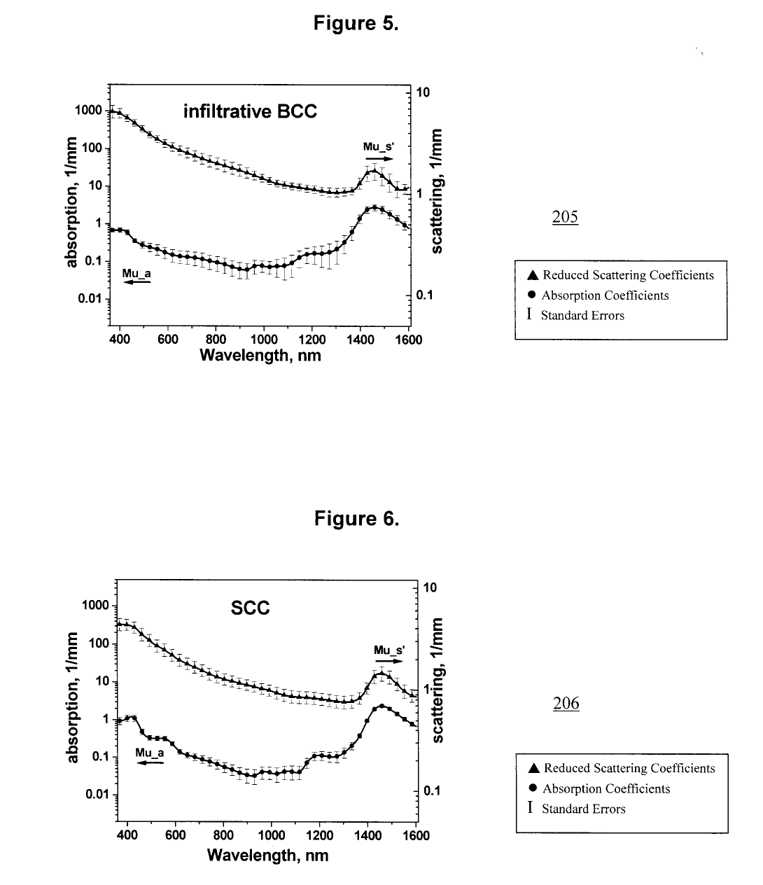

Referring now to FIGS. 1-9b, each figure has a legend 201-209b, respectively. The legends 201-209b include graphical references to further interpretation and understanding of the corresponding figures.

Averaged absorption and reduced scattering coefficients for each of the examined tissue types were calculated using the inverse Monte Carlo technique. The results are presented in FIGS. 1-9b. Absorption and scattering coefficients of healthy skin layers are shown in FIGS. 1-3 for epidermis, dermis, and subcutaneous fat, respectively. The graphs demonstrate that the scattering of normal skin layers decreases with the increasing wavelength. The steady decrease can be explained by the decrease of the contribution of Rayleigh scattering, whereas the contribution of Mie scattering increases with the increasing wavelength. For all the tissues investigated, an increase of scattering coefficient in the vicinity of the strong water absorption band around 1450 nm occurred.

The scattering of epidermis is noticeably higher than the scattering of dermis and subcutaneous fat in the entire wavelength range. It is known that optical properties of epidermis in the range 370-1200 nm are determined by melanin content. For this study, excisions taken from Caucasian subjects with fair skin were used. The content of melanin in the epidermis of these skin samples was comparatively low. However, the relative refractive index of melanin with respect to the surrounding medium is approximately 1.3. Therefore, light scattering in the epidermis is significantly higher than in other skin tissues. In the dermis, scattering is predominantly caused by collagen fibers and their associated small structures.

FIGS. 1-3 present absorption spectra of normal skin layers. In the visible wavelength range, melanin determines absorption in the epidermis. Absorption of melanin monotonously decreases with the increase of the wavelength. Therefore, the effect of melanin on epidermis absorption properties is more pronounced at shorter wavelengths. Hemoglobin dominates absorption properties of dermis and fat in the visible spectral range. Hemoglobin absorption peaks around 410 nm and 540 nm appear consistently in the spectra of dermis and fat, as all the specimens, except the epidermal, which contained some blood. Absorption of the epidermis, dermis and fat in the near-infrared region is determined by water and lipid content. In the proximity of 1200 nm, water and lipid absorption bands overlap. Therefore, this peak is more pronounced for the subcutaneous fat as compared to the epidermis and dermis. At the same time, the epidermis and dermis exhibit stronger absorption in the range from 1350 nm to 1600 nm.

In FIGS. 4-6, absorption and scattering properties of cancerous tissues are presented for nodular BCC, infiltrative BCC, and SCC, respectively. Scattering of all investigated nonmelanoma skin tumor types demonstrate qualitatively similar behavior. It gradually decreases with the increasing wavelength. Quantitatively, infiltrative basal cell carcinoma is characterized by a higher scattering coefficient in comparison with the scattering of nodular BCCs and SCC. The higher scattering coefficient of infiltrative BCC may be explained by its structural characteristics. Typically, these tumors have thin strands or cords of tumor cells extending into the surrounding highly scattering dermis. The scattering of squamous cell carcinomas is consistently lower than the scattering of both types of basal cell carcinomas in the entire wavelength range.

Absorptive properties of nonmelanoma skin cancers are determined by melanin and hemoglobin in the visible spectral range, and by water in the near infrared spectral range. Depositions of melanin often occur in the nonmelanoma tumors. The presence of this chromophore strongly affects absorption and scattering of the tumors. However, in general, the content of both melanin and hemoglobin in nonmelanoma skin cancers is highly variable. Therefore, the contrast based on the differences in melanin and hemoglobin content of the tumors as compared to normal skin tissues cannot be expected to occur reproducibly. It was found that on average, nodular BCCs contain less blood as compared to infiltrative BCCs and SCCs. It was also noted that infiltrative BCCs are characterized by a slightly higher absorption than squamous cell carcinomas in the range from 600 to 1600 nm. Absorption of nodular basal cell carcinomas is lower than that of infiltrative BCCs and SCCs.

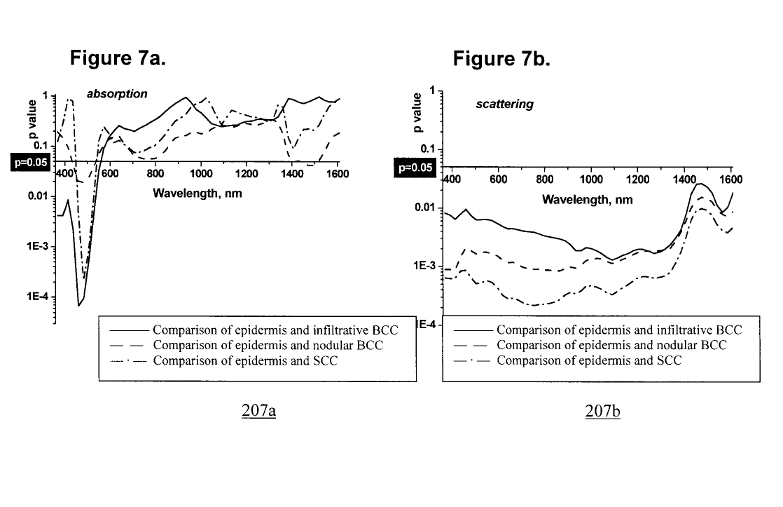

The spectral regions where the differences between cancerous tissues and healthy skin layers were statistically significant (p<0.05) can be clearly identified. P-plots presented in the FIGS. 7a-9b show the differences between absorption and scattering properties of the healthy epidermis, dermis, fat and nonmelanoma skin tumors, respectively. The spectral regions where optical properties of healthy and cancerous tissues differ significantly are listed in the FIG. 15.

In FIGS. 7a and 7b, the p-plots comparing absorption (FIG. 7a) and scattering (FIG. 7b) of epidermis to those of cancerous tissues are presented. As can be seen in FIG. 7a, the differences in absorption between the epidermis and all the types of cancer investigated are significant below 500 nm, in the vicinity of the hemoglobin Soret absorption band. As the blood content in cancerous samples is highly variable and may be affected by sample preparation technique, the identified significant differences in absorption are not likely to provide a reliable basis for tissue discrimination. At the same time, the differences in scattering between the epidermal and cancerous tissues were found to be significant in the complete spectral range investigated.

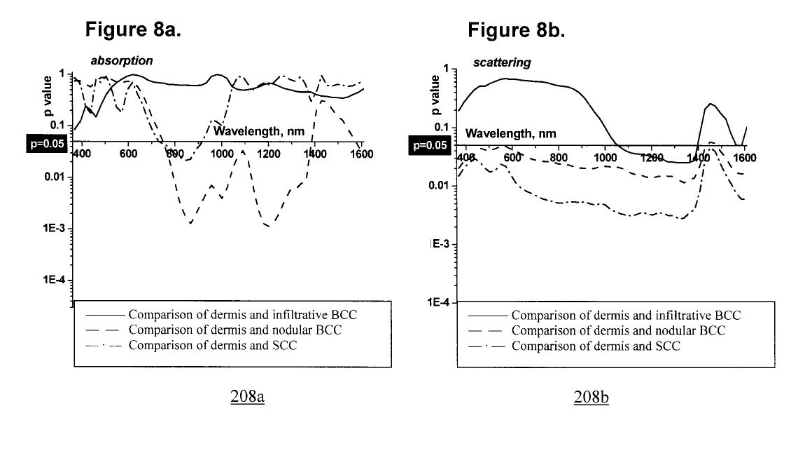

P-plots in FIGS. 8a and 8b show the differences in the absorption and scattering properties of the dermis and cancerous tissues, respectively. No significant differences were found in the absorption properties of the dermis and infiltrative BCC. This type of cancer is difficult to detect because, as was mentioned earlier, it is characterized by thin cancer cell strands invading the dermis. Therefore, the optical properties of infiltrative BCC are similar to those of the dermis. The differences in absorption of nodular BCCs and SCCs versus the dermis are significant in the wavelength range from 750 nm to 1380 nm and from 720 nm to 910 nm, respectively. Absorption in these cancer types was generally lower than in the dermis. P-plots comparing scattering properties of dermis-tumor pairs reveal that the scattering properties of dermis-nodular BCC and dermis-SCC exhibit significant differences in the complete wavelength range investigated. For infiltrative BCCs, the spectral range of significant differences was much narrower and covered the wavelengths between 1050 nm and 1400 nm.

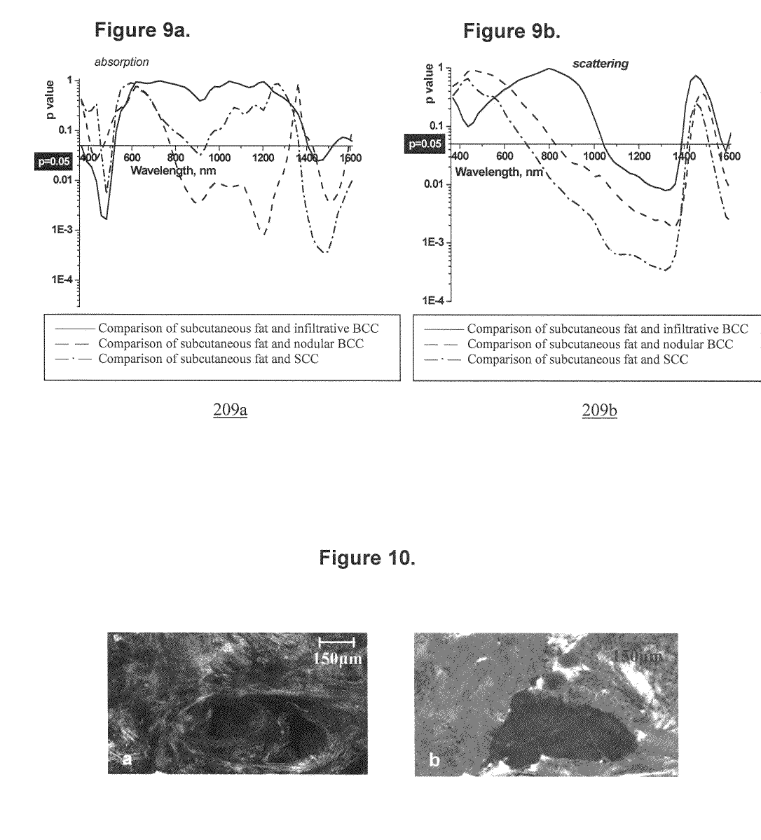

P-plots comparing absorption properties of subcutaneous fat and cancerous specimens (see FIG. 9a) demonstrate that for all tumor types there exist significant differences in the regions from 360 nm to 500 nm and from 1400 nm to 1500 nm. These two regions correspond to the absorption bands of blood and water, respectively. The results indicate that on average, cancerous tissue contains less blood and more water as compared to subcutaneous fat. Differences in scattering properties of subcutaneous fat-tumors pairs are significant for all cancer types in the wavelength range between 1050 nm and 1400 nm (see FIG. 9b).

The detected differences in the absorption properties of healthy skin-tumor pairs were provided by variations in the concentration of hemoglobin, melanin, and water. It has been reported previously that variations in water absorption can be used as a reliable parameter for distinguishing nonmelanoma skin cancers. Significant differences in water content were also found in all subcutaneous fat-tumor pairs. Water absorption in fat was consistently lower than that of cancerous specimens. However, other skin layers, i.e., epidermis and dermis, did not differ from cancer in terms of water content.

Statistical and comparative analysis of the differences in the optical properties of cancerous and normal skin revealed that all healthy skin layer-tumor pairs exhibited significant differences in scattering in the spectral range between 1050 nm and 1400 nm. Scattering in cancer specimens was substantially lower as compared to normal skin. At the same time, absorption properties of healthy and cancerous skin, with the exception of nodular BCC which exhibited significantly lower absorption than that of normal skin, did not differ substantially in this wavelength region. The results indicate that in the wavelength range between 370 nm and 1600 nm, there is no spectral region where the differences in absorption are significant for all healthy tissue—cancer pairs.

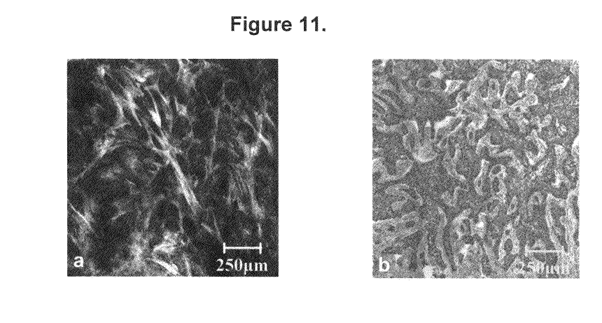

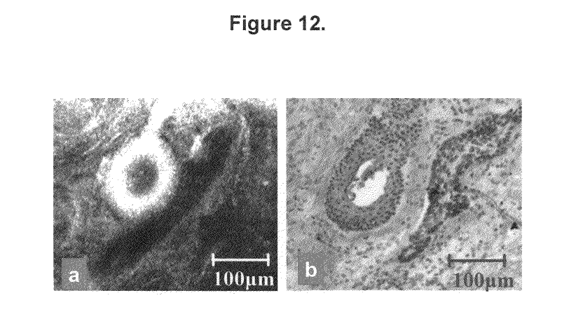

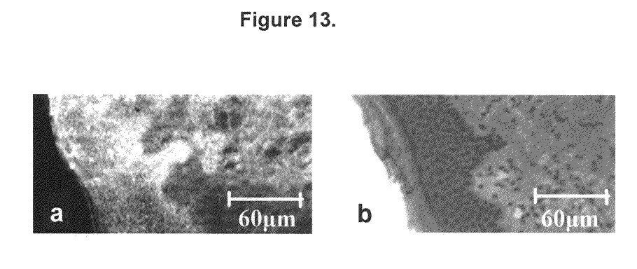

To confirm the findings several samples were imaged with different types of nonmelanoma skin cancer using a 1300-1350 nm reflectance confocal microscope. Example images of nonmelanoma skin cancers and adjacent healthy tissues are presented in FIGS. 10-13. The images in FIGS. 10-12 demonstrate that cancerous tissue is dark when compared to benign structures. Image specimens with superficial cancers are not shown. However, the image in FIG. 13 shows epidermis and surrounding dermis. Epidermis appears much brighter than dermis. Epidermis contains melanin, which exhibits significantly higher refractive index (approximately 1.7) as compared to the dermis (approximately 1.4). This suggests that detecting superficial tumors in the suggested wavelength range is also possible.

In accordance with the invention, an apparatus is provided for imaging cancer in which cancer tissue is contrasted against adjacent healthy tissue including illumination means configured and adapted to provide illumination light on a region of tissue. A Detection means is optically connected to the illumination means. The detection means are configured and adapted to detect reflected light from the tissue when the tissue is illuminated by the illumination means. A filtering means is optically coupled to the detection means. The filtering means are configured and adapted to filter at least one of the illumination light and the reflected light to pass only light within a range of about 1050 and about 1400 nm.

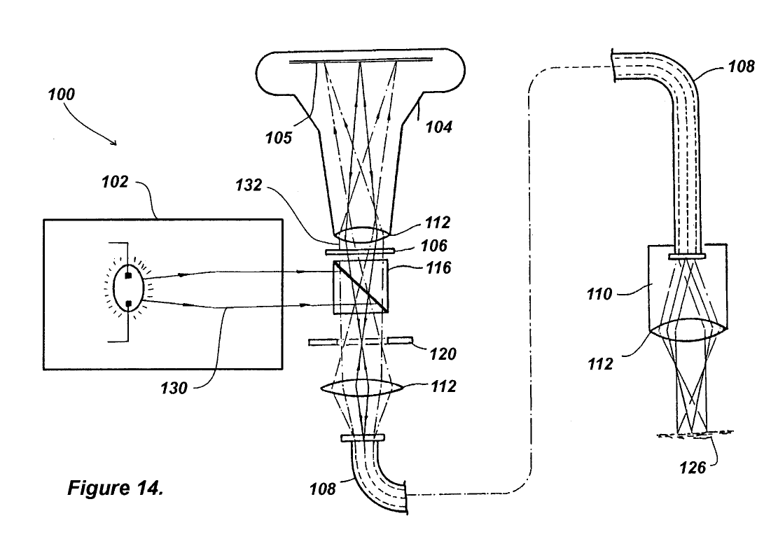

For purpose of explanation and illustration, and not limitation, a partial view of an exemplary embodiment of the apparatus for imaging cancer in accordance with the invention is shown in FIG. 14 and is designated generally by reference character 100.

For purposes of illustration and not limitation, as embodied herein and as depicted in FIG. 14, apparatus 100 is provided with an illumination means 102, which provides illumination light 130 for illuminating a region of tissue 126. Exemplary illumination means include (but are not limited to) lamps, lasers, LEDs, SLEDs, combinations thereof, or any other suitable device now known or later invented. Those skilled in the art will appreciate that any suitable light source can be used without departing from the spirit and scope of the invention.

In further accordance with the invention, a detection means 104 is provided for detecting reflected light from the region of tissue 126 illuminated by illumination means 102. Those skilled in the art will readily appreciate that any suitable detection means, including by way of example and not limitation, CCD 105 or other digital imaging devices, cameras using film, and other imaging devices as are known in the art. Additionally, those skilled in the art will readily appreciate that any type of elastic scattering optical imaging can be used, including point and line-scanning devices like confocal microscopy, snap-shot CCD imaging, optical coherence tomography imaging, or any other suitable type of imaging, without departing from the spirit and scope of the invention. Those skilled in the art will also appreciate that polarization imaging can also be sued without departing from the spirit and scope of the invention. For example, the illumination light can be polarized, the two reflected polarization components registered, and the resulting image processed as the difference of two polarizations or the difference divided by the sum multiplied by the normalization constant, as is known in the art of polarization imaging.

Detection means 104 and illumination means 102 are arranged so that illumination light 130 proceeds from illumination source 102 to ultimately illuminate a region of tissue 126. Beam splitter 116 reflects illumination light 130 toward tissue 126 and passes reflected light 132 returning from tissue 126 through to detection means 104. The region of tissue 126 reflects this light as reflected light 132, which ultimately reaches CCD 105 of detection means 104. Filtering means 106 filters reflected light 132 so that only light in the desired wavelength range passes into detection means 104.

High-contrast images of cancer tissue and adjacent healthy tissue can be produced with filtering means (or without filtering in the case of monochromatic lasers, LEDs, etc., for example) that pass light in the range from about 1050 and about 1400 nm, or any subrange thereof. Those skilled in the art will readily appreciate that within this range, narrow ranges can also be used to produce images in which cancer tissue is contrasted against healthy adjacent tissue. By way of example and not limitation, a range of 1300 nm to 1350 nm can provide high contrast images, however, those skilled in the art will readily appreciate that any subset of ranges in the range of about 1040 nm to about 1400 nm can be used without departing from the spirit and scope of the invention.

While FIG. 14 shows filtering means 106 filtering only reflected light 132, those skilled in the art will readily appreciate that a wide variety of other configurations are possible. A filter can be placed to filter only illumination light 130, and it is even possible to combine the filter and illumination means 102 as a source that emits illumination light only in the desired wavelength range (such as in a narrowband gas or diode laser, tunable laser, narrow band LEDs, narrow band SLEDs, or any other suitable device now known or later invented that can emit as a monochromatic source). A monochromatic illumination source can also be used in conjunction with additional filtering means. It is also possible to locate a filtering means 106 so that both illumination light 130 and reflected light 132 are filtered at the same location. Filtering means 106 can be combined with other components, such as one or more lenses 112, beam splitter 116, light screen 120, illumination means 104, etc. In some configurations it is advantageous to have multiple filtering means 106. Those skilled in the art will readily appreciate other suitable configurations of filtering means 106 that are possible without departing from the spirit and scope of the invention. Moreover, those skilled in the art will recognize that it is also possible to use a narrow band filter, bandpass filter, monochroimator, interference filters, liquid crystal filter, or other suitable filters now known or later invented with lasers, lamps, LEDs, SLEDs or other suitable illumination sources without departing from the spirit and scope of the invention.

In accordance with one embodiment of the apparatus of the invention, illumination means 102 and detection means 104 can be combined as a confocal imaging section, as in a confocal microscope. A plurality of lenses 112 and at least one light screen 120, or other suitable scanning device can be used to focus and scan images of tissue 126 into detection means 104. A flexible optical coupling element 108, such as an optical fiber or a bundle of optical fibers, conveys light between the confocal imaging section 102/104 and probe 110. Probe 110 casts illumination light 130 on tissue 126 and receives reflected light 132 back into coupling element 108 for imaging. Those skilled in the art will readily appreciate that flexible optical coupling element 108 and probe 110 are optional, however, they provide the advantages of mobility allowing non-invasive and minimally invasive in vivo imaging as in endoscopic and laparoscopic applications to a wide variety of surface tissues and also internal tissues. Element 108 can also be a coherent or incoherent bundle of optical fibers. In the case of an incoherent bundle, means for descrambling reflected light 132 must also be provided in order to produce images, as is known in the art.

Additionally, those skilled in the art will appreciate that the systems and method for imaging cancer described above can be used in conjunction with wide-field imaging devices. An exemplary wide-field imaging device useable in conjunction with the invention is described in U.S. patent application Ser. No. 11/823,610 by Yaroslavsky et al., filed Jun. 28, 2007, which is incorporated herein by reference in its entirety. Those skilled in the art will readily appreciate how to practice the invention with this wide-field imaging device, or any other suitable wide-field imaging device now known or later invented, without departing from the spirit and scope of the invention.

While the apparatus of the invention has been described above in the context of a confocal microscope configured to operate endoscopically, those skilled in the art will readily appreciate that the high contrast imaging of cancer tissue in the wavelengths described above can be accomplished on a wide variety of devices, including but not limited to cameras, microscopes, and other cancer imaging devices, without departing from the spirit and scope of the invention. Those skilled in the art will readily appreciate that any suitable device that registers elastic scattering can be used without departing from the spirit and scope of the invention.

In accordance with another aspect of the invention, a method of for imaging cancer in which cancer tissue is contrasted against adjacent healthy tissue is provided. The method includes the steps of illuminating a region of tissue with illumination light, and light reflected from the region such that filtered light is only in the optical bandwidth between about 1050 and about 1400 nm or in any subrange of this range, and registering an image based on the reflected filtered light.

For purposes of illustration and not limitation, as embodied herein and as depicted in FIG. 14, illumination light (e.g., 132) is cast from illumination means (e.g., 102) onto a region of tissue of interest (e.g., 126). Light reflected from the tissue is detected and constructed into an image by a suitable detection means (e.g., 104, 105). At some point along the path from the illumination means to the detection means, at least one of the illumination light or reflected light is filtered, as described above, to pass light within an optical bandwidth of about 1050 nm to about 1400 nm. Those skilled in the art will readily appreciate that within this range there are many smaller bandwidths in which the optical bandwidth is one in which cancerous tissue and healthy tissue have absorption coefficients that are approximately equal, and in which cancerous tissue has a higher or lower scattering coefficient than healthy tissue, causing cancerous tissue to appear brighter or darker than healthy tissue, in accordance with the invention. It is also possible to operate in a bandwidth in which both absorption and scattering coefficients are significantly different for cancerous and healthy tissue, such as is the case for nodular BCC, which exhibits lower absorption than healthy tissue from 1050 nm to 1400 nm, as described above. It is possible, for example, to filter the reflected light and/or the illumination light to pass only light in any subrange between 1050 nm and 1400 nm. It is also possible that the method can be carried out on a confocal microscope, such as apparatus 100 described above. It is possible to use an endoscopic configuration to make possible in vivo inspection of surface tissues as well as tissues within a body. However, those skilled in the art will readily appreciate that the method can also be performed on a wide variety of other devices without departing from the spirit and scope of the invention.

The methods and systems of the present invention, as described above and shown in the drawings, provide for a method and apparatus for imaging cancer with superior properties including providing high contrast between cancer tissue and adjacent healthy tissue. It will be apparent to those skilled in the art that various modifications and variations can be made in the device and method of the present invention without departing from the spirit or scope of the invention. Thus, it is intended that the present invention include modifications and variations that are within the scope of the appended claims and their equivalents.

What is claimed is:

1. An apparatus for imaging cancerous and precancerous tissue in which the cancer affected tissue is contrasted against adjacent normal tissue, the apparatus comprising:
a) illumination means configured and adapted to provide illumination light on a region of tissue; and
b) detection means optically coupled to the illumination means, the detection means being configured and adapted to detect reflected light from the tissue when the tissue is illuminated by the illumination means, wherein the reflected light is substantially within a range of about 1050 and about 1400 nm.

2. An apparatus as recited in claim 1, further comprising filtering means optically coupled to the detection means, the filtering means being configured and adapted to filter at least one of the illumination light and the reflected light to pass only light within the range.

3. An apparatus as recited in claim 1, wherein the illumination means is selected from the group consisting of a laser, a LED, a SLED, and combinations thereof.

4. An apparatus as recited in claim 1, wherein the filtering means is configured to pass only light within a wavelength range in which cancer affected tissue and normal tissue have absorption coefficients that are approximately equal, and in which cancer affected tissue has a different scattering coefficient than normal tissue, causing cancer affected tissue to appear different than normal tissue.

5. An apparatus as recited in claim 4, wherein the filtering means is configured to pass only light within a wavelength range in which cancer affected tissue has a lower scattering coefficient than normal tissue, causing cancer affected tissue to appear brighter than normal tissue.

6. An apparatus as recited in claim 4, wherein the filtering means is configured to pass only light within a wavelength range in which cancer affected tissue has a higher scattering coefficient than normal tissue, causing cancer affected tissue to appear darker than normal tissue.

7. An apparatus as recited in claim 2, wherein the filtering means is configured and adapted to filter at least one of the illumination light and the reflected light to pass only light within a range of about 1300 and about 1350 nm.

8. An apparatus as recited in claim 1, wherein the illumination means and detection means are combined in a confocal imaging section.

9. An apparatus as recited in claim 1, further comprising at least one flexible optical coupling element optically coupled between the confocal imaging section and a remote probe that is insertable into a body.

10. An apparatus as recited in claim 9, wherein the at least one flexible optical coupling element is a coherent bundle of optical fibers.

11. An apparatus as recited in claim 9, wherein the at least one flexible optical coupling element is an incoherent bundle of optical fibers, and wherein the imaging section includes means for descrambling the reflected light received through the incoherent bundle to form an image.

12. An apparatus as recited in claim 2, wherein the filtering means is combined with the illumination means as a narrowband laser.

13. A microscope for imaging cancer in which cancer tissue is contrasted against adjacent healthy tissue, the microscope comprising:
a) at least one probe section configured and adapted to illuminate a region of tissue of interest;
b) an illuminating section for generating illumination light for the at least one probe section;
c) an imaging section for constructing images from reflected light remitted from the region of interest;
d) filtering means in optical communication with the imaging section, the filtering means being configured and adapted to filter the illumination light and the reflected light to pass only light in the optical bandwidth between about 1300 to about 1350 nm; and
e) at least one flexible optical coupling element optically coupled between the imaging section and the probe section.

14. A microscope as recited in claim 13, wherein the microscope is a confocal microscope and wherein the illumination section and the imaging section are combined into a confocal imaging section for generating illumination light for the at least one probe section and for constructing images from reflected light remitted from the region of interest.

15. A microscope as recited in claim 13, wherein the probe section is a remote probe insertable into locations within a body in place of an endoscope.

16. A microscope as recited in claim 13, wherein the at least one flexible optical coupling element is a coherent bundle of optical fibers.

17. A microscope as recited in claim 13, wherein the at least one flexible optical coupling element is an incoherent bundle of optical fibers, and wherein the imaging section includes means for descrambling the reflected light from the incoherent bundle to form an image.

18. A microscope as recited in claim 13, wherein the filtering means is combined with the illumination means as a light source of a type selected from a list consisting of: narrowband gas laser, narrowband diode laser, tunable laser, and narrowband LED.

19. A microscope as recited in claim 13, wherein the filtering means is a filter of a type selected from a list consisting of: bandpass filter, narrow band filter, monochroimator, interference filter, and liquid crystal filter.

20. A method of imaging cancer in which cancer tissue is contrasted against adjacent healthy tissue, the method comprising:
a) illuminating a region of tissue with illumination light;
b) filtering at least one of the illumination light and light reflected from the region such that filtered light is only in the optical bandwidth between about 1050 and about 1400 nm; and
c) registering an image based on the reflected filtered light.

21. A method of imaging cancer as recited in claim 20, wherein the filtering step includes passing only light within a wavelength range in which cancerous tissue and healthy tissue have absorption coefficients that are approximately equal, and in which cancerous tissue has a higher scattering coefficient than healthy tissue, causing cancerous tissue to appear darker than healthy tissue.

22. A method of imaging cancer, as recited in claim 20, where the step of illuminating includes polarizing the illumination light, and wherein the step of registering includes registering two reflected polarization components to be processed into an image.

23. A method of imaging cancer as recited in claim 20, wherein the steps of illuminating and registering are performed in a confocal imaging section of a confocal microscope.

24. An method of imaging cancer as recited in claim 21, wherein the step of illuminating includes transmitting illumination light between the confocal imaging section and a remote probe that is insertable into a body.
